(19) United States
(12) Patent Application Publication
(10) Pub. No.: US 2022/0306678 A1
(43) Pub. Date: Sep. 29, 2022

(54) IMPROVED IN VITRO TRANSCRIPTION PURIFICATION PLATFORM

(71) Applicant: ARCTURUS THERAPEUTICS, INC., San Diego, CA (US)

(72) Inventors: Maher ALAYYOUBI, San Diego, CA (US); Jared Henry DAVIS, San Diego, CA (US)

(21) Appl. No.: 17/619,882

(22) PCT Filed: Jun. 17, 2020
(86) PCT No.: PCT/US2020/038126

§ 371 Date: Dec. 16, 2021

Related U.S. Application Data
(60) Provisional application No. 62/864,430, filed on Jun. 20, 2019.

(51) Int. Cl. C07H 21/02; C12N 15/10; B01D 15/38; B01D 15/42

(52) U.S. Cl. CPC C07H 21/02; C12N 15/101; B01D 15/3847; B01D 15/426

(57) ABSTRACT

Provided herein are methods for purification of RNA from a sample. The methods include obtaining a first sample including double stranded RNA in a loading buffer, loading the sample onto a ceramic hydroxyapatite column, washing the column with wash buffer, and eluting the column with an elution buffer to create an eluate.

### BACKGROUND

Messenger RNA (mRNA) can be used as a therapeutic agent in the treatment of a variety of diseases. Administering mRNA compositions requires mRNA products of acceptable purity, however high levels of double stranded RNA (dsRNA) impurities can result from the in vitro preparation of RNA, including mRNA. These dsRNA impurities can generate an immune response and reduce the efficacy of the mRNA treatment.

Purification processes including nuclease enzymes and/or purification columns can be effective, however these systems have proven difficult to scale up to manufacturing large batches of mRNA. New methods of purification and dsRNA removal are needed that can be scaled to large batch sizes of mRNA, can provide better yields, can provide higher purity including dsRNA removal, and are efficient and robust.

### BRIEF SUMMARY

In view of the foregoing, there is a need for compositions and methods that address the diversity in the process of transcript purification and removal of double stranded RNA. The present disclosure addresses this need, and provides additional benefits

In an aspect, provided herein are methods for purification of RNA from a sample. The methods include obtaining a first sample including double stranded RNA in a loading buffer, loading the sample onto a ceramic hydroxyapatite column, washing the column with wash buffer, and eluting the column with an elution buffer to create an eluate.

### BRIEF DESCRIPTION OF THE DRAWINGS

FIG. 1 shows that mRNA binds to ceramic hydroxyapatite (CHT) column and elutes with a NaPi gradient. The chromatogram is of a run in which the column is run in bind-elute mode and the sample is in NaPi without additives. The solid line is Absorbance at 260 nm. The dashed line is conductivity.

FIG. 2 shows that CHT fails to separate ssRNA (single stranded RNA) and dsRNA (double stranded RNA) with a regular NaPi gradient. Dot blot assay results for load and peak fractions fr15, fr16, and fr17 from CHT II column are shown in duplicate in the left panel. The dot blot quantification is shown in the bar graph in the right panel. As can be seen in the comparison of the load and fraction 15, there is some efficacy of this method as there is less dsRNA in the peak fractions compared to load.

FIGS. 3A-3B show that dsRNA density is 20 times less in peak compared to control when sample is run in 15% ethanol. FIG. 3A shows the chromatogram for parameters in which the column is run in bind-elute mode and the sample is in 15% ethanol without NaCl. The solid line is Absorbance at 260 nm. The dashed line is conductivity. FIG. 3B shows dot blot assay results for load and peak fraction from CHT II column for Run1 in 15% ethanol in the top panel. The dot blot quantification is shown in the bar graph in the bottom panel. As can be seen in the comparison of the load and peak fraction, there is 20 times less dsRNA in the peak fraction compared to load.

FIGS. 4A-4B show that dsRNA is at 5% in peak compared to load when sample is run in 15% ethanol with 100 mM NaCl. FIG. 4A shows the chromatogram for parameters in which the column is run in bind-elute mode and the sample is in 15% ethanol with 100 mM NaCl. The solid line is Absorbance at 260 nm. The dashed line is conductivity. FIG. 4B shows dot blot assay results for load and peak from CHT II column for Run2 in 15% ethanol with 100 mM NaCl in the top panel. The dot blot quantification is shown in the bar graph in the bottom panel. As can be seen in the comparison of the load and peak fraction, the dsRNA is at 4.45% in the peak fraction compared to the load.

FIGS. 5A-5B show that dsRNA density is 7 times less in peak compared to control when sample is run in 4% acetonitrile. FIG. 5A shows the chromatogram for parameters in which the column is run in bind-elute mode and the sample is in 4% acetonitrile. The solid line is Absorbance at 260 nm. The dashed line is conductivity. FIG. 5B shows dot blot assay results for load and peak fractions from CHT II column for Run1 in 4% acetonitrile in the top panel. The dot blot quantification is shown in the bar graph in the bottom panel. As can be seen in the comparison of the load and peak fractions, the amount of dsRNA in the peak fractions is approximately 14% of the load.

FIGS. 6A-6B show that dsRNA density is only 3 times less in peak compared to control when sample is run in 4% acetonitrile with NaCl. FIG. 6A shows the chromatogram for parameters in which the column is run in bind-elute mode and the sample is in 4% acetonitrile. and 100 mM NaCl. The solid line is Absorbance at 260 nm. The dashed line is conductivity. FIG. 6B shows dot blot assay results in top panel for load and peak fractions from CHT II column for Run2 in 4% acetonitrile with 100 mM NaCl. The dot blot quantification is shown in the bar graph in the bottom panel. As can be seen in the comparison of the load and peak fractions, the amount of dsRNA in the peak fractions is approximately 33% of the load.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

### Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by (+) or (−) 10%, 5%, 1%, or any subrange or sub-value there between. Preferably, the term “about” when used with regard to a dose amount means that the dose may vary by +/−10%.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention. In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited are not changed by the presence of more than that which is recited, but excludes prior art embodiments.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

As used herein, the term “nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In some embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non-limiting examples, of nucleosides include cytidine, uridine, adenosine, guanosine, and thymidine. The term “nucleotide” refers, in the usual and customary sense, to a single unit of a polynucleotide, i.e., a monomer, but may also refer to nucleoside monomer having one to three phosphate or phosphorothioate groups. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Modified ribonucleotides include diaminopurine, N⁶-methyl-2-aminoadenosine, N⁶-methyladenosine, 5-carboxycytidine, 5-formyl-cytidine, 5-hydroxycytidine, 5-hydroxymethylcytidine, 5-methoxycytidine, 5-methylcytidine, N⁴-methylcytidine, thienoguanosine, 5-carboxymethylesteruridine, 5-formyluridine, 5-hydroxymethuluridine, 5-methoxyoxyuridine, N¹-methylpseudouridine, 5-methyluridine, and pseudouridine.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methylcytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos, unlocked nucleic acids (UNA), or locked nucleic acids (LNA) as known in the art). Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, the term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

As used herein, the term “transcription” refers to the first step of gene expression, in which a particular segment of DNA is copied into RNA (especially mRNA) by the enzyme RNA polymerase. During transcription, a DNA sequence is read by an RNA polymerase, which produces a complementary, antiparallel RNA strand called a primary transcript. The stretch of DNA transcribed into an RNA molecule is called a “transcription unit” and encodes at least one gene. If the gene encodes a protein, the transcription produces messenger RNA (mRNA); the mRNA, in turn, serves as a template for the protein's synthesis through translation. Alternatively, the transcribed gene may encode for non-coding RNA such as microRNA, ribosomal RNA (rRNA), transfer RNA (tRNA), or enzymatic RNA molecules called ribozymes. Overall, RNA helps synthesize, regulate, and process proteins; it therefore plays a fundamental role in performing functions within a cell.

As used herein, the term “restriction enzyme” or “restriction endonuclease” refers to an enzyme that cleaves DNA into fragments at or near specific recognition sites within the molecule known as restriction sites. Restrictions enzymes are one class of the broader endonuclease group of enzymes. Restriction enzymes are commonly classified into five types, which differ in their structure and whether they cut their DNA substrate at their recognition site, or if the recognition and cleavage sites are separate from one another. To cut DNA, all restriction enzymes make two incisions, once through each sugar-phosphate backbone (i.e. each strand) of the DNA double helix.

As used herein, the terms “ceramic hydroxyapatite column” and “CHT column” refer to a purification column comprised of ceramic hydroxyapatite (CHT). CHT is a spherical, macroporous form of hydroxyapatite that overcomes the limitations of the crystalline material and allows use in industrial-scale columns. Separation protocols originally developed on crystalline hydroxyapatite can be transferred directly to the ceramic material with little or no modification. CHT ceramic hydroxyapatite retains the unique separation properties of crystalline hydroxyapatite, but can be used reproducibly for several hundred cycles at high flow rates and in large columns.

### Methods of Use

In an aspect, provided herein are methods for reducing double stranded RNA (dsRNA) in a transcribed RNA product. In embodiments, the transcribed RNA product includes mRNA. The methods include obtaining a first sample comprising double stranded RNA in a loading buffer, loading the sample onto a ceramic hydroxyapatite column, washing the column with wash buffer; and eluting the column with an elution buffer to create an eluate.

In embodiments, the eluate comprises less than 50% of the double stranded RNA in the first sample. In embodiments, the eluate comprises less than 40% of the double stranded RNA in the first sample. In embodiments, the eluate comprises less than 30% of the double stranded RNA in the first sample. In embodiments, the eluate comprises less than 20% of the double stranded RNA in the first sample. In embodiments, the eluate comprises less than 10% of the double stranded RNA in the first sample. In embodiments, the eluate comprises less than 1% of the double stranded RNA in the first sample.

In embodiments, the first sample is obtained from an in vitro transcription reaction.

In embodiments, the first sample is obtained from an affinity column, a hydrophobic interaction column, an anionic exchange column, a cationic exchange column, a reverse phase column, a mixed phase column, a precipitation treatment, or a combination thereof. In embodiments, the first sample is obtained from an affinity column. In embodiments, the first sample is obtained from a hydrophobic interaction column. In embodiments, the first sample is obtained from an anionic exchange column. In embodiments, the first sample is obtained from a cationic exchange column. In embodiments, the first sample is obtained from a reverse phase column. In embodiments, the first sample is obtained from a mixed phase column. In embodiments, the first sample is obtained from a precipitation treatment. In embodiments, the first sample is obtained from a combination of one or more of an affinity column, a hydrophobic interaction column, an anionic exchange column, a cationic exchange column, a reverse phase column, a mixed phase column, and a precipitation treatment.

In embodiments, loading the sample onto a ceramic hydroxyapatite column is conducted at room temperature. In embodiments, loading the sample onto a ceramic hydroxyapatite column is conducted at a temperature of about 15° C. to about 30° C. In embodiments, loading the sample onto a ceramic hydroxyapatite column is conducted at a temperature of about 15° C. In embodiments, loading the sample onto a ceramic hydroxyapatite column is conducted at a temperature of about 16° C. In embodiments, loading the sample onto a ceramic hydroxyapatite column is conducted at a temperature of about 17° C. In embodiments, loading the sample onto a ceramic hydroxyapatite column is conducted at a temperature of about 18° C. In embodiments, loading the sample onto a ceramic hydroxyapatite column is conducted at a temperature of about 19° C. In embodiments, loading the sample onto a ceramic hydroxyapatite column is conducted at a temperature of about 20° C. In embodiments, loading the sample onto a ceramic hydroxyapatite column is conducted at a temperature of about 21° C. In embodiments, loading the sample onto a ceramic hydroxyapatite column is conducted at a temperature of about 22° C. In embodiments, loading the sample onto a ceramic hydroxyapatite column is conducted at a temperature of about 23° C. In embodiments, loading the sample onto a ceramic hydroxyapatite column is conducted at a temperature of about 24° C. In embodiments, loading the sample onto a ceramic hydroxyapatite column is conducted at a temperature of about 25° C. In embodiments, loading the sample onto a ceramic hydroxyapatite column is conducted at a temperature of about 26° C. In embodiments, loading the sample onto a ceramic hydroxyapatite column is conducted at a temperature of about 27° C. In embodiments, loading the sample onto a ceramic hydroxyapatite column is conducted at a temperature of about 28° C. In embodiments, loading the sample onto a ceramic hydroxyapatite column is conducted at a temperature of about 29° C. In embodiments, loading the sample onto a ceramic hydroxyapatite column is conducted at a temperature of about 30° C.

In embodiments, the loading buffer includes salt. In embodiments, the loading buffer includes sodium phosphate. In embodiments, the loading buffer includes sodium chloride.

In embodiments, the loading buffer includes about 1 to about 50 mM, about 2 to about 40 mM, about 3 to about 30 mM, about 4 to about 20 mM or about 5 to about 10 mM sodium phosphate. In embodiments, the loading buffer includes about 10 mM, about 15 mM, about 20 mM, about 25 mM, about 30 mM, about 35 mM, about 40 mM, about 45 mM, or about 50 mM sodium phosphate. In embodiments, the loading buffer includes about 1 mM, about 2 mM, about 3 mM, about 4 mM, about 5 mM, about 6 mM, about 7 mM, about 8 mM, about 9 mM, or about 10 mM sodium phosphate. The amount may be any value or subrange within the recited ranges, including endpoints.

In embodiments, the loading buffer includes about 50 to about 1000 mM, about 100 to about 950 mM, about 150 to about 900 mM, about 200 to about 850 mM, about 250 to about 800 mM, about 300 to about 750 mM, about 350 to about 700 mM, about 400 to about 650 mM, or about 450 to about 600 mM sodium chloride. In embodiments, the loading buffer includes about 50 mM, about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, or about 1000 mM sodium chloride. The amount may be any value or subrange within the recited ranges, including endpoints.

In embodiments, the method includes equilibrating the column prior to loading the sample. In embodiments, equilibrating the column includes adding wash buffer.

In embodiments, the equilibration buffer includes sodium phosphate. In embodiments, the equilibration buffer includes sodium chloride.

In embodiments, the equilibration buffer includes about 1 to about 50 mM, about 2 to about 40 mM, about 3 to about 30 mM, about 4 to about 20 mM or about 5 to about 10 mM sodium phosphate. In embodiments, the equilibration buffer includes about 10 mM, about 15 mM, about 20 mM, about 25 mM, about 30 mM, about 35 mM, about 40 mM, about 45 mM, or about 50 mM sodium phosphate. In embodiments, the equilibration buffer includes about 1 mM, about 2 mM, about 3 mM, about 4 mM, about 5 mM, about 6 mM, about 7 mM, about 8 mM, about 9 mM, or about 10 mM sodium phosphate. The amount may be any value or subrange within the recited ranges, including endpoints.

In embodiments, the equilibration buffer includes about 50 to about 1000 mM, about 100 to about 950 mM, about 150 to about 900 mM, about 200 to about 850 mM, about 250 to about 800 mM, about 300 to about 750 mM, about 350 to about 700 mM, about 400 to about 650 mM, or about 450 to about 600 mM sodium chloride. In embodiments, the equilibration buffer includes about 50 mM, about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, or about 1000 mM sodium chloride. The amount may be any value or subrange within the recited ranges, including endpoints.

In embodiments, the wash buffer includes a C₁-C₅ alcohol. In embodiments, the C₁-C₅ alcohol is selected from methanol, ethanol, propanol, butanol, and pentanol. In embodiments, the wash buffer includes methanol. In embodiments, the wash buffer includes ethanol. In embodiments, the wash buffer includes propanol. In embodiments, the wash buffer includes butanol. In embodiments, the wash buffer includes pentanol.

In embodiments, the wash buffer includes about 10% to about 30% ethanol in water. In embodiments, the wash buffer includes about 15% to about 25% ethanol in water. In embodiments, the wash buffer includes about 10% ethanol in water. In embodiments, the wash buffer includes about 15% ethanol in water. In embodiments, the wash buffer includes about 20% ethanol in water. In embodiments, the wash buffer includes about 25% ethanol in water. In embodiments, the wash buffer includes about 30% ethanol in water. The amount may be any value or subrange within the recited ranges, including endpoints.

In embodiments, the elution buffer includes a soluble phosphate salt selected from sodium phosphate and potassium phosphate. In embodiments, the elution buffer includes sodium phosphate In embodiments, the elution buffer includes potassium phosphate.

In embodiments, the elution buffer includes about 50 to about 1000 mM, about 100 to about 950 mM, about 150 to about 900 mM, about 200 to about 850 mM, about 250 to about 800 mM, about 300 to about 750 mM, about 350 to about 700 mM, about 400 to about 650 mM, or about 450 to about 600 mM sodium phosphate. In embodiments, the elution buffer includes about 50 mM, about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, or about 1000 mM sodium phosphate. The amount may be any value or subrange within the recited ranges, including endpoints.

In embodiments, the elution buffer includes about 50 to about 1000 mM, about 100 to about 950 mM, about 150 to about 900 mM, about 200 to about 850 mM, about 250 to about 800 mM, about 300 to about 750 mM, about 350 to about 700 mM, about 400 to about 650 mM, or about 450 to about 600 mM potassium phosphate. In embodiments, the elution buffer includes about 50 mM, about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, or about 1000 mM potassium phosphate. The amount may be any value or subrange within the recited ranges, including endpoints.

In embodiments, each of the loading buffer, the wash buffer, and the elution buffer includes one or more of urea, guanidine chloride, and acetonitrile. In embodiments, each of the loading buffer, the wash buffer, and the elution buffer includes urea. In embodiments, each of the loading buffer, the wash buffer, and the elution buffer includes guanidine chloride. In embodiments, each of the loading buffer, the wash buffer, and the elution buffer includes acetonitrile.

In embodiments, the acetonitrile is about 10-30% acetonitrile in water. In embodiments, the acetonitrile is about 15-25% acetonitrile in water. In embodiments, the acetonitrile is about 10% acetonitrile in water. In embodiments, the acetonitrile is about 15% acetonitrile in water. In embodiments, the acetonitrile is about 20% acetonitrile in water. In embodiments, the acetonitrile is about 25% acetonitrile in water. In embodiments, the acetonitrile is about 30% acetonitrile in water. The amount may be any value or subrange within the recited ranges, including endpoints.

In embodiments, the mRNA comprises one or more modified ribonucleotides. In embodiments, the ribonucleotides include modified ribonucleotides. In embodiments, the modified ribonucleotides include one or more selected from diaminopurine, N⁶-methyl-2-aminoadenosine, N⁶-methyladenosine, 5-carboxycytidine, 5-formyl-cytidine, 5-hydroxycytidine, 5-hydroxymethylcytidine, 5-methoxycytidine, 5-methylcytidine, N⁴-methylcytidine, thienoguanosine, 5-carboxymethylesteruridine, 5-formyluridine, 5-hydroxymethyluridine, 5-methoxyoxyuridine, N¹-methylpseudouridine, 5-methyluridine, and pseudouridine. In embodiments, the modified ribonucleotides include diaminopurine. In embodiments, the modified ribonucleotides include N⁶-methyl-2-aminoadenosine. In embodiments, the modified ribonucleotides include N⁶-methyladenosine. In embodiments, the modified ribonucleotides include 5-carboxycytidine. In embodiments, the modified ribonucleotides include 5-formyl-cytidine. In embodiments, the modified ribonucleotides include 5-hydroxycytidine. In embodiments, the modified ribonucleotides include 5-hydroxymethylcytidine. In embodiments, the modified ribonucleotides include 5-methoxycytidine. In embodiments, the modified ribonucleotides include 5-methylcytidine. In embodiments, the modified ribonucleotides include N⁴-methylcytidine. In embodiments, the modified ribonucleotides include thienoguanosine. In embodiments, the modified ribonucleotides include 5-carboxymethylesteruridine. In embodiments, the modified ribonucleotides include 5-formyluridine. In embodiments, the modified ribonucleotides include 5-hydroxymethyluridine. In embodiments, the modified ribonucleotides include 5-methoxyoxyuridine. In embodiments, the modified ribonucleotides include N′-methylpseudouridine. In embodiments, the modified ribonucleotides include 5-methyluridine. In embodiments, the modified ribonucleotides include pseudouridine.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: CHT Column Separation of ssRNA and dsRNA Using Sodium Phosphate Gradient

In this study, experiments were conducted to investigate the use of ceramic hydroxyapatite for the separation of ssRNA and dsRNA at room temperature.

Hydroxyapatite (Ca₅(PO₄)₃OH)₂ (HA) is a form of calcium phosphate used in the chromatographic separation of biomolecules. Sets of five calcium doublets (C-sites) and pairs of —OH containing phosphate triplets (P-sites) are arranged in a repeating geometric pattern. (See Biorad CHT Ceramic hydroxyapatite instruction manual).

HA resins are mixed mode resins and have been proven efficient in purification of both RNA and DNA oligonucleotides, which interact with the column's C-sites. This study focused mainly on ceramic hydroxyapatite type II resin as a chromatography method for the separation of ssRNA from dsRNA.

Double stranded RNA (dsRNA) levels were visualized using a “Dot Blot” assay, an immunoblot assay which uses the dsRNA-specific antibody MJ2.

Methods: Using the Biorad-Bioscale Mini CHT type II column (40 μm cartridge, 5 mL), a sample including RNA was loaded onto the column. The load was 5 ml in 500 mM NaCl, 10 mM sodium phosphate (NaPi), pH 7.0. The Resin Challenge was 0.2 mg/mL. The Flow rate was set at 5 mL/min, 1 CV/min (Column Volumes per minute). The column was washed with 10 mM NaPi, pH 7.0 wash buffer (Buffer A) and eluted with 500 mM NaPi, pH 7.0 (Buffer B). The elution used the following Run Method: 3CV 0% B wash, 15 CVs 00-100% B gradient, 3CVs 100% B wash, 3 CV 0.1 N NaOH wash.

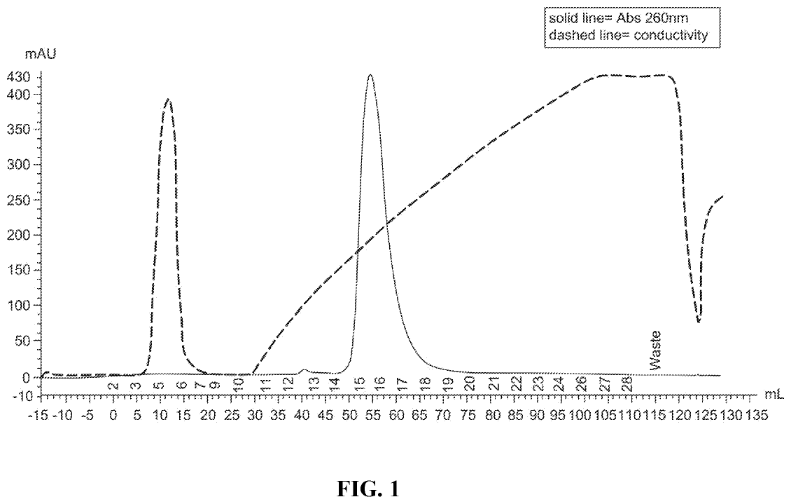

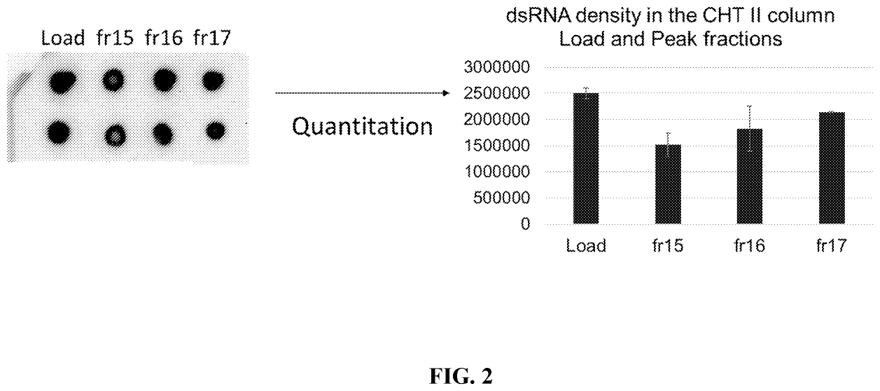

As shown in FIG. 1, mRNA binds to the CHT column and elutes with a sodium phosphate gradient. Dot blot assay results for load and peak from CHT II column and quantitation of the dot blot assays are shown in FIG. 2. The data show some efficacy in separation of ssRNA and dsRNA with a regular sodium phosphate gradient (comparison of fraction 15 to load).

dsRNA is more saturated in the right shoulder of the CHT II peak, indicating that dsRNA binds tighter to CHT column than ssRNA. Use of step gradients and shallower gradients were unsuccessful in further separating the ssRNA from the dsRNA.

### Example 2: CHT Column Separation of ssRNA and dsRNA Using 15% Ethanol

Experiments were conducted to evaluate use of ethanol in purification of ssRNA using a CHT column. Data presented here show that ethanol used at 15% can also help in the separation of ssRNA from dsRNA on a ceramic hydroxyapatite column type II.

Methods: Using the Biorad-Bioscale Mini CHT type II column (40 μm cartridge, 5 mL), a sample including RNA, previously purified using an affinity column was further treated. The load was in 10 mM NaPi, pH 7.0, 15% ethanol, and the Resin Challenge was 1 mg/ml. The flow rate was set at 5 ml/min, 1 CV/min. The column was washed with a wash buffer of 10 mM NaPi, pH 7.0, 15% ethanol (Buffer A) and eluted with a Buffer B gradient where buffer B was 350 mM NaPi, pH 7.0, 15% ethanol. The following Run Method was used: 3 CV 0% B wash, 15 CVs 00-100% B gradient, 3 CVs 100% B wash, 3 CV 0.1 N NaOH wash.

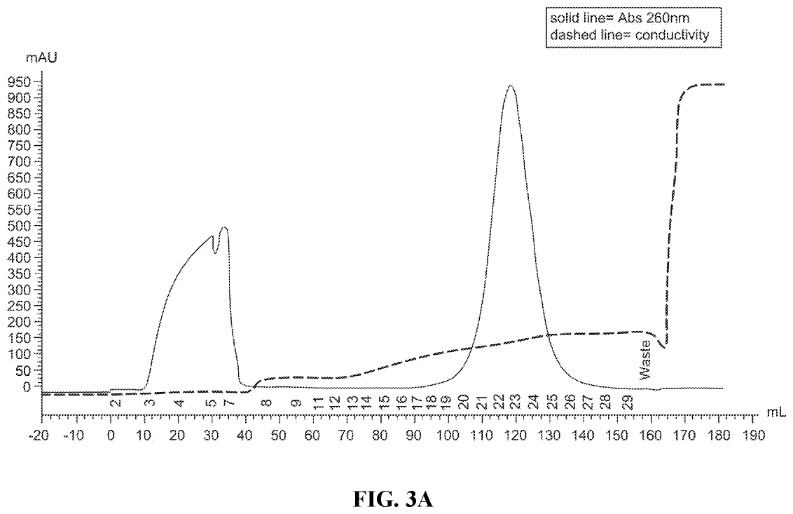

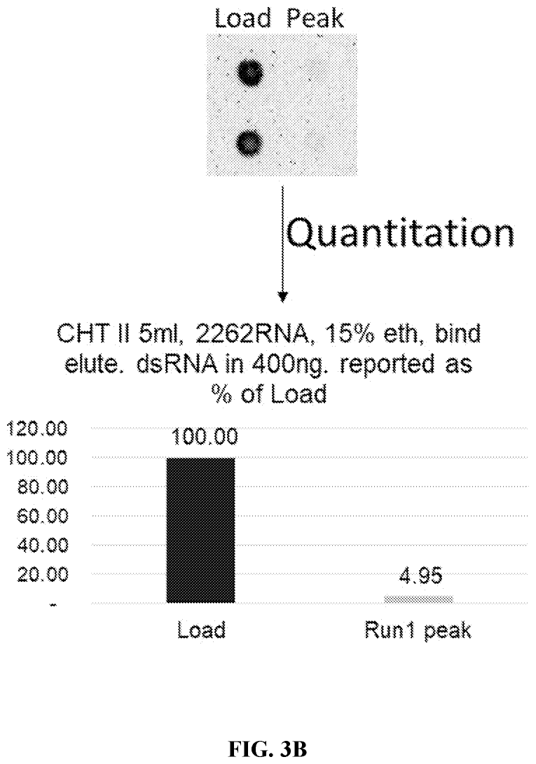

As shown in FIG. 3A, dsRNA density is 20 times less in peak compared to the dsRNA density in the load. CHT dot blot assay results for load and peak from CHT II column and quantitation of the dot blot assays are shown in FIG. 3B. The data show improved efficacy in separation of ssRNA and dsRNA with the peak from the column having 5% of the dsRNA density compared to the load. However, the recovery of ssRNA was poor (55%).

In a second set of experiments, the addition of sodium chloride was tested.

Methods: Using the Biorad-Bioscale Mini CHT type II column (40 μm cartridge, 5 mL), a sample including RNA previously purified using an affinity column was further treated. The load was 30 ml in 10 mM NaPi, 100 mM sodium chloride (NaCl), pH 7.0, 15% ethanol, with a Resin Challenge of 1 mg/ml. The flow rate was set at 5 ml/min, 1 CV/min. The column was washed with a wash buffer of 10 mM NaPi, pH 7.0, 15% ethanol (Buffer A) and eluted with Buffer B of 350 mM NaPi, pH 7.0, 15% ethanol. The flow rate was set at 5 ml/min, 1 CV/min. The following Run Method was used: 3 CV 0% B wash, 15 CVs 00-100% B gradient, 3 CVs 100% B wash, 3 CV 0.1 N NaOH wash.

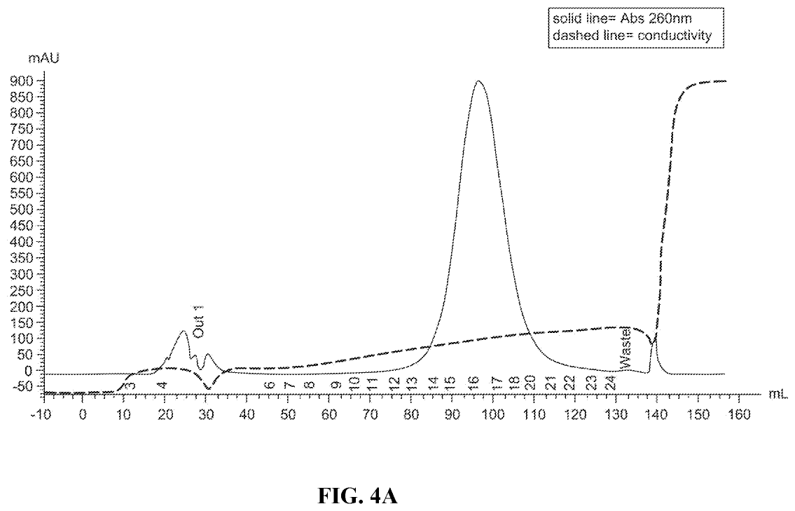

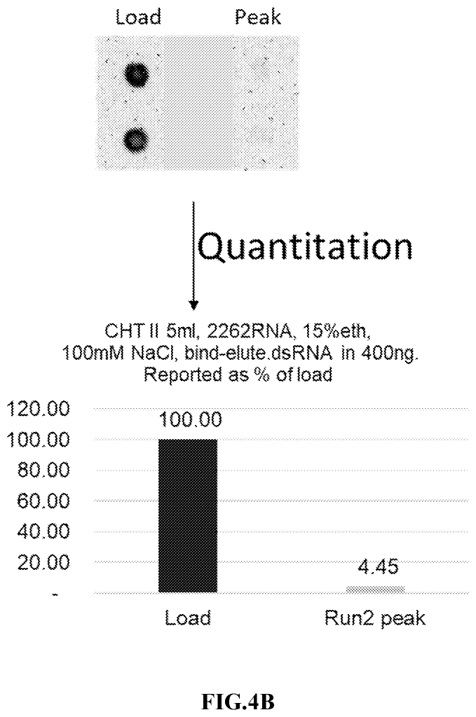

As shown in FIG. 4A, addition of 100 mM NaCl to the load buffer increased the capacity of the column. CHT dot blot assay results for load and peak from CHT II column and quantitation of the dot blot assays are shown in FIG. 4B. The data show further improvement in separation of ssRNA and dsRNA and an increase in the recovery of ssRNA to 70%.

### Example 3: CHT Column Separation of ssRNA and dsRNA Using 4% Acetonitrile

Experiments were conducted to evaluate use of acetonitrile in purification of ssRNA using CHT.

Methods: Using the Biorad-Bioscale Mini CHT type II column (40 μm cartridge, 5 mL), a sample including RNA previously purified on an affinity column was further treated by adding a 10 mL load in 10 mM NaPi, pH 7.0, 4% acetonitrile, and a Resin Challenge of 1 mg/mL. The flow rate was set at 5 mL/min, 1 CV/min. The column was washed with a wash buffer of 10 mM NaPi, pH 7.0, 4% acetonitrile (Buffer A) and eluted with Buffer B of 250 mM NaPi, pH 7.0, 4% acetonitrile. The following Run Method was used: 3 CV 0% B wash, 15 CVs 00-100% B gradient, 3 CVs 100% B wash, 3 CV 0.1 N NaOH wash.

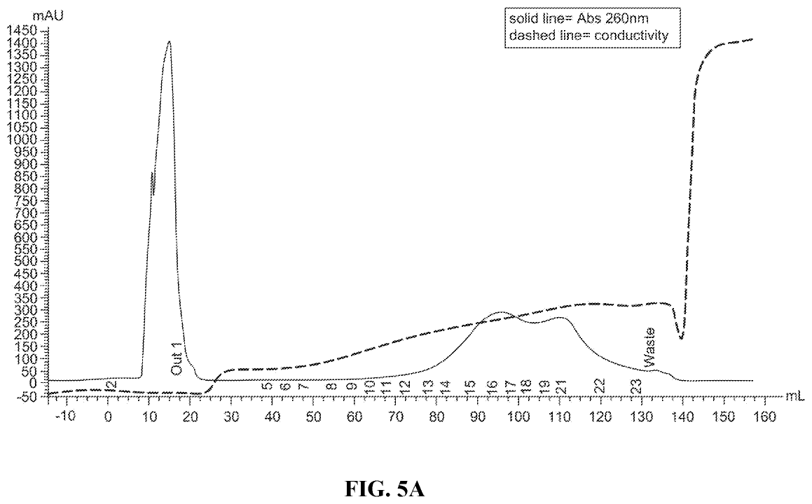

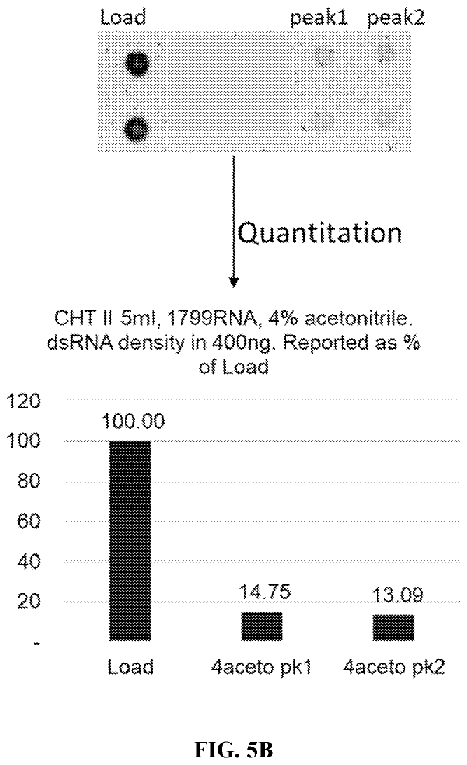

As shown in FIG. 5A, the dsRNA density is 7 times less in the peak when 4% acetonitrile is used in the wash and elution buffers, compared to the dsRNA density of the sample load. Dot blot assay results for load and peak from CHT II column and quantitation of the dot blot assays are shown in FIG. 5B.

In a second set of experiments, the addition of sodium chloride was tested.

Methods: Using the Biorad-Bioscale Mini CHT type II column (40 μm cartridge, 5 mL), a sample including RNA previously purified using an affinity column was further treated by adding a 10 mL load, in 10 mM NaPi, 100 mM sodium chloride (NaCl), pH 7.0, 4% acetonitrile, and a Resin Challenge of 1 mg/mL. The flow rate was set at 5 mL/min, 1 CV/min. The column was washed with a wash buffer of 10 mM NaPi, pH 7.0, 4% acetonitrile (Buffer A) and eluted with Buffer B of 250 mM NaPi, pH 7.0, 4% acetonitrile. The following Run Method was used: 3 CV 0% B wash, 15 CVs 00-100% B gradient, 3 CVs 100% B wash, 3 CV 0.1 N NaOH wash.

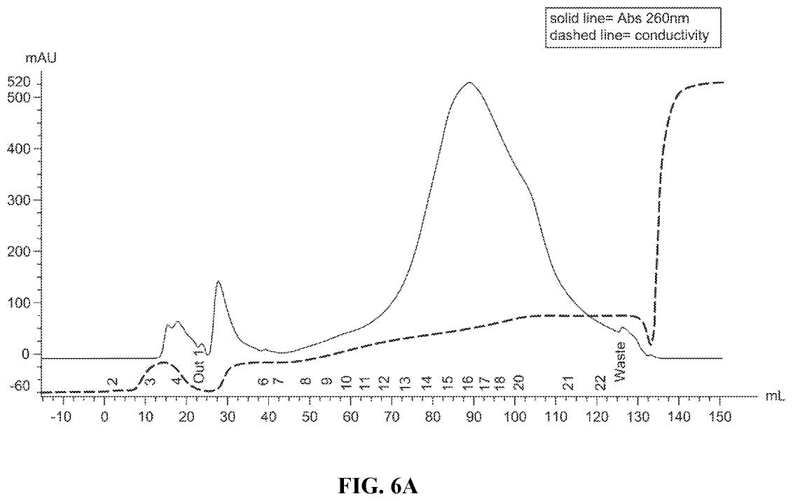

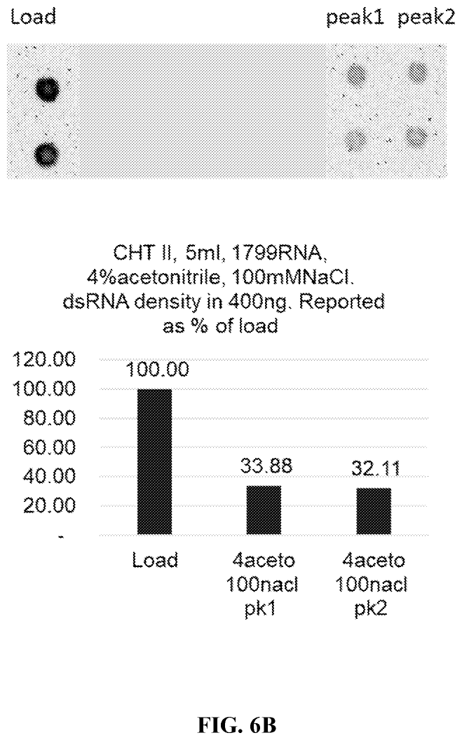

As shown in FIG. 6A, the dsRNA density is 3 times less in the peak compared to the dsRNA density of the sample load when 4% acetonitrile and NaCl is used in the wash and elution buffers. Dot blot assay results for load and peak from CHT II column and quantitation of the dot blot assays are shown in FIG. 6B.

These experiments showed that 4% acetonitrile increases the separation of the ssRNA and dsRNA on ceramic hydroxyapatite column type II. When 100 mM NaCl is added to the sample with 4% acetonitrile, the capacity of the column increased.

Further, 15% ethanol and 4% acetonitrile proved useful in the separation ssRNA and dsRNA on a CHT II column compared to the control run of Example 1 that included the phosphate gradient with no additives.

1. A method for reducing double stranded RNA (dsRNA) in a transcribed RNA product, the method comprising:
a. obtaining a sample comprising dsRNA in a loading buffer;
b. loading the sample onto a ceramic hydroxyapatite column;
c. washing the column with wash buffer; and
d. eluting the column with an elution buffer to create an eluate.

2. The method of claim 1, wherein the eluate comprises less than 50% of the dsRNA in the sample.

3. The method of claim 1, wherein the eluate comprises less than 40% of the dsRNA in the sample.

4. The method of claim 1, wherein the eluate comprises less than 30% of the dsRNA in the sample.

5. The method of claim 1, wherein the eluate comprises less than 20% of the dsRNA in the sample.

6. The method of claim 1, wherein the eluate comprises less than 10% of the dsRNA in the sample.

7. The method of claim 1, wherein the eluate comprises less than 1% of the dsRNA in the sample.

8. The method of claim 1, wherein the sample is obtained from an affinity column, a hydrophobic interaction column, an anionic exchange column, a reverse phase column, a mixed phase column, or a precipitation treatment.

9. The method of claim 1, wherein the sample and the eluate comprise mRNA.

10. The method of claim 9, wherein the mRNA comprises one or more modified ribonucleotides.

11. The method of claim 10, wherein the one or more modified ribonucleotides is selected from diaminopurine, N⁶-methyl-2-aminoadenosine, N⁶-methyladenosine, 5-carboxycytidine, 5-formyl-cytidine, 5-hydroxycytidine, 5-hydroxymethylcytidine, 5-methoxycytidine, 5-methylcytidine, N⁴-methylcytidine, thienoguanosine, 5-carboxymethylesteruridine, 5-formyluridine, 5-hydroxymethuluridine, 5-methoxyoxyuridine, N¹-methylpseudouridine, 5-methyluridine, and pseudouridine.

12. The method of claim 1, wherein step (b) is conducted at room temperature.

13. The method of claim 1, wherein the loading buffer comprises a salt.

14. The method of claim 13, wherein the salt is sodium chloride.

15. The method of claim 13, wherein the loading buffer comprises 50-1000 mM sodium chloride.

16. The method of claim 1, wherein the wash buffer comprises a C₁-C₅ alcohol.

17. The method of claim 16, wherein the wash buffer comprises ethanol.

18. The method of claim 17, wherein the wash buffer comprises 10% to 30% ethanol in water.

19. The method of claim 1, wherein the elution buffer comprises a soluble phosphate salt selected from sodium phosphate and potassium phosphate.

20. The method of claim 1, wherein each of the loading buffer, the wash buffer, and the elution buffer comprises one or more of urea, guanidine chloride, and acetonitrile.

21. The method of claim 20, wherein the acetonitrile is 10-30% acetonitrile in water.
